(19) United States
(12) Patent Application Publication
(10) Pub. No.: US 2025/0197348 A1
(43) Pub. Date: Jun. 19, 2025

(54) Acid Degradable Solid Lipid Nanoparticles

(71) Applicant: The Regents of the University of California, Oakland, CA (US)

(72) Inventors: Michael R. Stentzel, Berkeley, CA (US); Jie Li, Berkeley, CA (US); Hesong Han, Berkeley, CA (US); Niren Murthy, Berkeley, CA (US)

(73) Assignee: The Regents of the University of California, Oakland, CA (US)

(21) Appl. No.: 19/056,724

(22) Filed: Feb. 18, 2025

Related U.S. Application Data
(63) Continuation of application No. PCT/US22/41785, filed on Aug. 28, 2022.

(51) Int. Cl. C07C 401/00; A61K 47/69; A61K 48/00; C07C 209/32; C07C 211/02; C07C 235/08; C12N 15/88

(52) U.S. Cl. CPC C07C 401/00; A61K 47/6929; A61K 48/0008; C07C 209/32; C07C 211/02; C07C 235/08; C12N 15/88

(57) ABSTRACT

Acid degradable solid lipid nanoparticles comprise PEG conjugated to cholesterol via an acid degradable linkage comprising an azide-benzaldehyde acetal.

### CROSS-REFERENCES TO RELATED APPLICATIONS

This application is a continuation of PCT/US22/41785, filed Aug. 28, 2022, the disclosures of which are hereby incorporated by reference in its entirety for all purposes.

### GOVERNMENT SUPPORT CLAUSE

This invention was made with government support under grant numbers NS115599 and EB029320 from the National Institutes of Health. The government has certain rights in the invention.

### INTRODUCTION

Solid lipid nanoparticles have tremendous potential for delivering mRNA and have the potential to treat a wide variety of diseases. SLNs contain a PEGylated lipid, which is generally in the 1-5% range and is needed to maintain SLN stability, size, tissue diffusion and lower toxicity. However, excessive PEGylation also results in lower cell uptake and endosomal disruption. This paradox has limited the efficacy of SLNs, and is termed the “PEG dilemma”. Acid degradable PEG-lipids have great potential for overcoming the PEG dilemma, but have been challenging to develop due to the synthetic challenges associated with working with acetals and their instability at pH 7.4.

### SUMMARY OF THE INVENTION

The invention provides compositions comprising acid degradable solid lipid nanoparticles, components thereof, including PEG-conjugated to cholesterol via an acid degradable linkage comprising an azide-benzaldehyde acetal, and related compositions, intermediates and methods of making and using the compositions.

In an aspect the invention provides compositions comprising PEG-conjugated to cholesterol via an acid degradable linkage comprising an azide-benzaldehyde acetal.

In an aspect the invention provides a composition comprising azide-benzaldehyde acetal cholesterol.

In embodiments:
the acetal is conjugated to PEG, wherein the azide-benzaldehyde acetal provides an acid degradable linkage between the PEG and the cholesterol;
the azide is reduced to amine (e.g. prior to injection, for rapid hydrolysis);

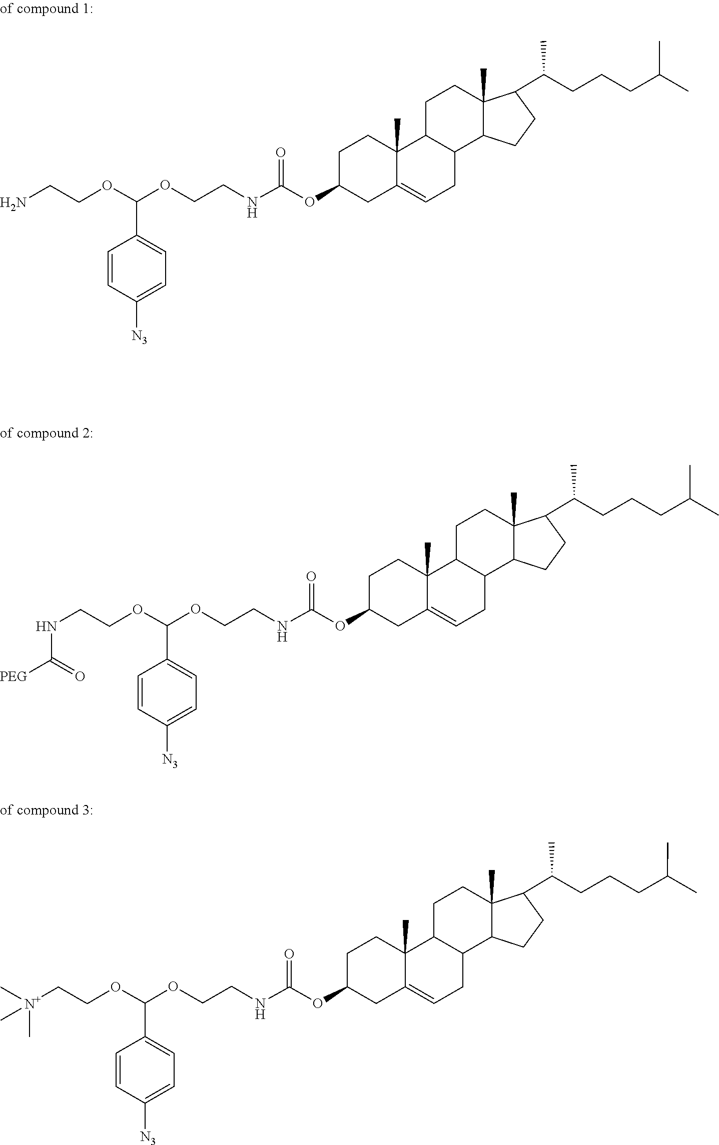

or a salt thereof.
formulated into solid lipid nanoparticles (SLNs) further comprising a nucleic acid, such as RNA or DNA, preferably encoding a therapeutic protein, vaccine antigen, or gene editing enzyme(s); and/or
formulated into solid lipid nanoparticles (SLNs), and/or stored, and/or then reduced prior to biological use, to generate an amino acetal.

In an aspect the invention provides a method of use, comprising transfecting with a disclosed composition, a tissue or organ, such as muscle, lung, spleen, liver and blood.

In embodiments:
further comprising detecting a resultant delivery of nucleic acid in the tissue or organ.

In an aspect the invention provides methods, processes, compositions and systems disclosed herein, including those of the figures.

The invention encompasses all combinations of the particular embodiments recited herein, as if each combination had been laboriously recited.

### BRIEF DESCRIPTION OF THE DRAWINGS

FIG. 1. Acid degradable PEGylated solid lipid nanoparticles for mRNA delivery.

FIG. 2. Synthesis of Acid degradable PEG-Lipid.

FIG. 3. LNPs: Lipid Materials in Formulations: mPEG2k-ALL-Chol: PEGylated lipid; DOPE: “Helper” lipid (stability and delivery); DOTAP: Endosomal disruption; D-Lin (D-Lin-MC3-DMA): Transfectionl; Chol: Stability

FIG. 4. LNPs made with 1 deliver luciferase mRNA to lung tissue; bar graph shows radiance (p/sec/cm²/sr), the number of photons per second that are leaving a square centimeter of tissue and radiating into a solid angle of one steradian (sr).

FIG. 5. Synthesis of a cationic lipid that transforms into a neutral lipid in the endosome.

FIG. 6. LNPs made with compound 3 deliver luciferase mRNA to lung tissue; bar graph shows radiance (p/sec/cm²/sr).

FIG. 7. LNPs made with 3 deliver CRE mRNA to lung tissue: Ai9 mice treated with LNPs made from compound 3 (left); Ai9 mice treated with saline (right).

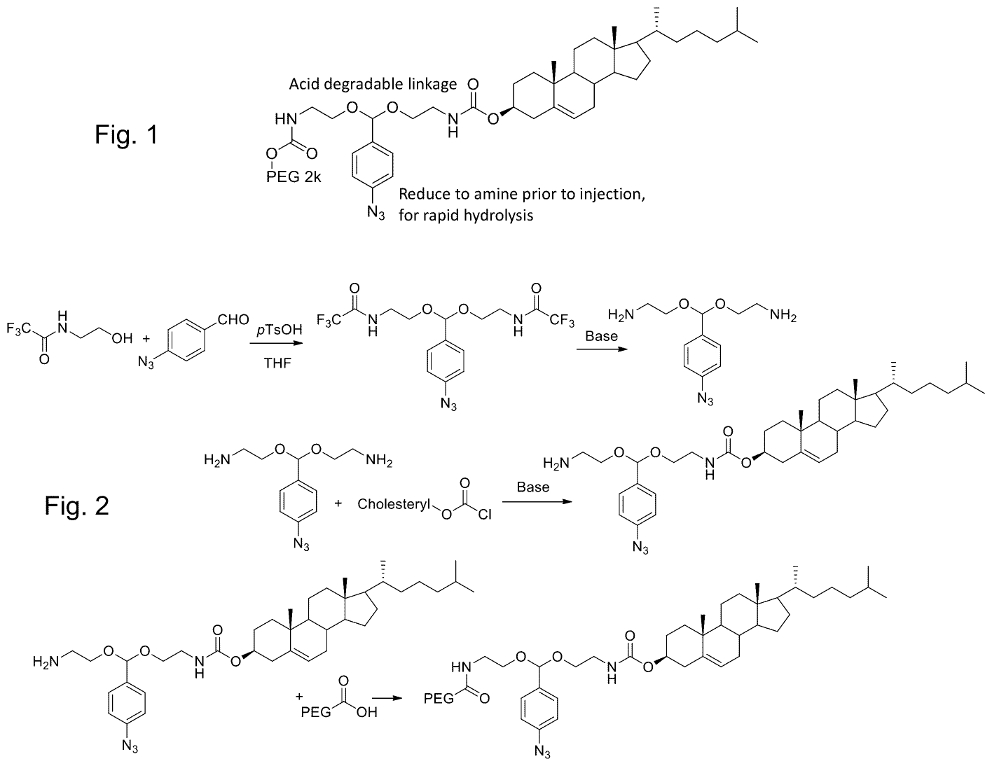

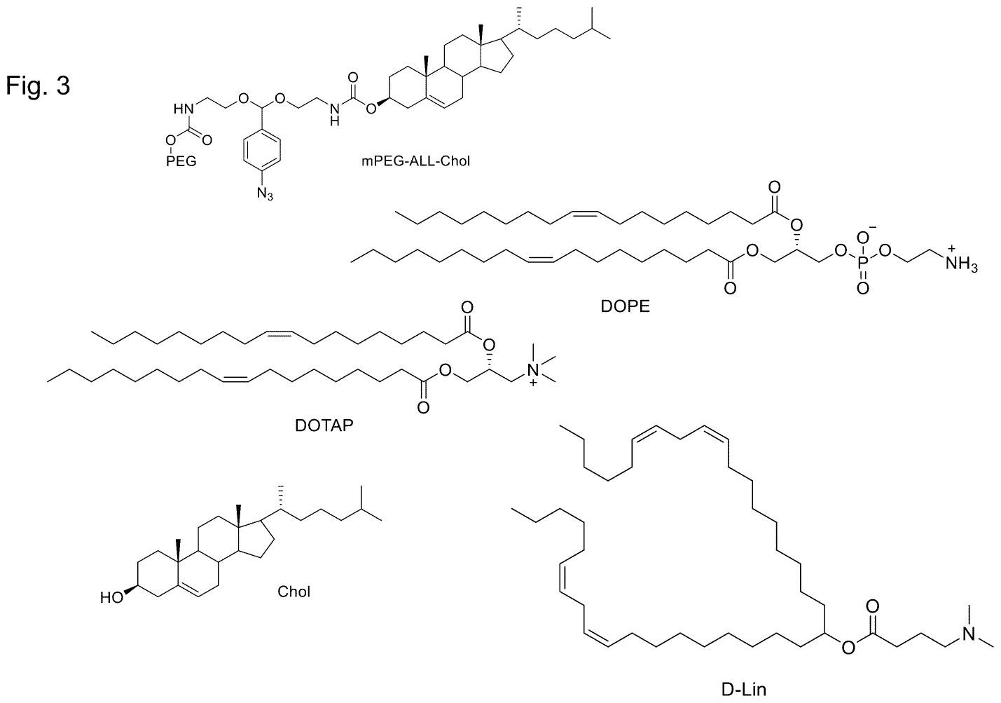

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We disclose compositions comprising PEG-conjugated to cholesterol via an acid degradable linkage composed of an azide-benzaldehyde acetal and uses. The compositions overcome the PEG dilemma and allow SLNs to be PEGylated with mole ratios up to 50%. The azide-benzaldehyde acetal, has its azide in the para position, and generates stable acetals with a t ½ of >1000 minutes at pH 7.4. These PEG-acetals can be formulated into SLNs, and stored, and then reduced prior to biological use, to generate an amino acetal that has t½<60 minutes at pH 7.4 and several minutes at pH 5.0. The ultra-PEGylated lipids were efficient at transfecting a variety of organs, including the muscle, the lung, spleen and liver and were also able to transfect the blood. The invention provides numerous applications of the azide-benzaldehyde acetal linker, given its unique ability to be stable prior to reductive activation, including medical applications; for example, it can be used for delivering mRNA to a variety of organs, such as the heart, the liver, lungs, spleen, brain, for vaccine development, gene editing and numerous others.

### EXAMPLES

We performed experiments demonstrating:
a) Lipid nanoparticles with acid degradable PEG-lipids (2 kD PEG) transfect mRNA in HELA cells efficiently
b) Lipid nanoparticles with acid degradable PEG-lipids (2 kD PEG) transfect mRNA in HEK cells efficiently
c) Lipid nanoparticles with acid degradable PEG-lipids (1 kD PEG) transfect mRNA in HELA cells efficiently
d) Lipid nanoparticles with acid degradable PEG-lipids (500 Da PEG) transfect mRNA in HELA cells efficiently
e) Lipid nanoparticles with acid degradable PEG-lipids (2 kD PEG) transfect muscle tissue efficiently
f) Lipid nanoparticles with acid degradable PEG-lipids (2 kD PEG) transfect mRNA systemically in mice after intravenous injection
g) Lipid nanoparticles with acid degradable PEG-lipids (2 kD PEG) transfect cells in the blood
h) Lipid nanoparticles with acid degradable PEG-lipids transfect mRNA into brain tissue after an intracranial injection
i) Acid degradable LNPs can be engineered to transfect lung tissue with specificity; and
j) Acid degradable cationic lipids transfect spleen tissue with specificity after an intravenous injection.

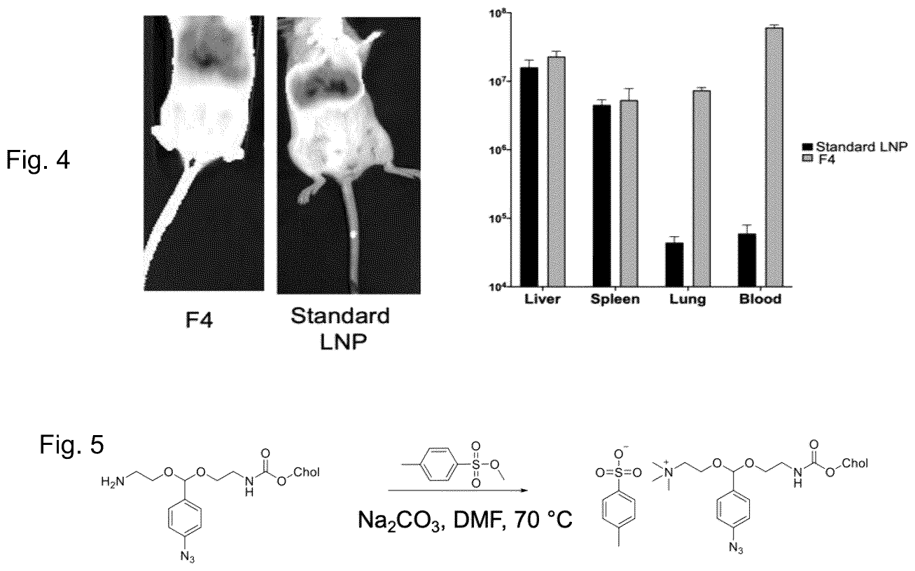

Exemplary Data. LNPs made with compound 1 tolerate very high levels of PEG and this influences their tissue tropism. In particular, high levels of PEGylation reduce uptake by liver macrophages and allow LNPs access to non-liver organs. We verified the tissue tropism of LNPs made with compound 1 and compared them against the standard LNP formulation. LNPs were made with F-Luc mRNA (size=1929 bases) and were injected into mice at a concentration of 10 μg per mouse via the tail vein and imaged 4 hours later in an IVIS imaging machine. In addition, the mice were sacrificed, and the organs were isolated and imaged ex vivo in an IVIS imaging machine. FIG. 4 demonstrates that LNPs made with compound 1 can efficiently deliver mRNA to the lungs after intravenous injection. The standard LNP formulation, generated high luciferase expression levels in the liver and spleen, reaching 10⁷ photons/second, but had very low luciferase expression in the lung (0.5×10⁵ photons/second), which was similar to background levels. In contrast, LNPs made with 1 generated high luciferase expression levels in the lung and had expression levels close to 10⁷ photons/second, which was 100 times greater than the luciferase expression levels generated by the standard LNP formulation.

Compound 3 is a cationic lipid that transforms into neutral lipids in endosomes. The ACE linker has tremendous versatility with regards to the types of acid degradable lipids it can generate, due to the ability to make compound 1 on a large scale. We investigated if the ACE linker could be used to synthesize cationic lipids, which fragment in endosomes and transform into neutral lipids. Cationic lipids are the major cause of toxicity generated by LNPs and several second-generation degradable cationic lipids have been synthesized, which have ester groups that will hydrolyze in cells. Degradable lipids, such MC3-DLin, have lower toxicity than non-degradable cationic lipids, however, they still generate a large cytokine response in patients. A key limitation of ester linkages is their uncertain hydrolysis timescale, as the cellular concentration of enzymes that degrade these lipids are unknown. The ACE linker has the potential to generate cationic lipids that rapidly degrade in endosomes, due to its rapid pH 6.0 hydrolysis kinetics, and should degrade orders of magnitude faster than MC3-DLin. We synthesized compound 3, following the synthetic strategy shown in FIG. 5, it was purified via silica gel chromatography and analyzed via H-NMR and mass spectrometry. 1H NMR (400 MHz, Chloroform-d) δ 7.75 (d, J=7.7 Hz, 2H), 7.36 (d, J=8.5 Hz, 2H), 7.14 (d, J=7.9 Hz, 2H), 7.01 (d, J=8.4 Hz, 2H), 5.51 (s, 1H), 5.42-5.31 (m, 2H), 5.52-5.40 (m, 1H), 4.04-3.94 (m, 2H), 3.94-3.87 (m, 1H), 3.65-3.55 (m, 1H), 3.54-3.46 (m, 1H), 3.37 (s, 10H), 3.20-3.09 (m, 1H), 2.34 (s, 3H), 2.30-2.18 (m, 2H), 2.04-1.92 (m, 2H), 1.92-1.75 (m, 3H), 1.63-1.04 (m, 12H), 1.4-1.94 (m, 5H), 0.91 (d, J=6.5 Hz, 3H), 0.87 (d, J=1.9 Hz, 3H), 0.85 (d, J=1.9 Hz, 2H), 0.67 (s, 3H). C₄₂H₆₈N₅O₄ [M]+706.5266, found 706.5263.

Compound 3 contains a quaternary amine and the ACE linker. Upon reduction and acidification it rapidly hydrolyzes in endosomes and releases its positive charge. LNPs made with compound 3 have low toxicity because their cationic lipids are transformed into neutral lipids (cholesterol) and not persist. In addition, LNPs made with compound 3 also efficiently disrupt endosomes via a colloid osmotic mechanism, wherein each lipid is converted into three molecules in the endosome, which osmotically destabilize the endosome.

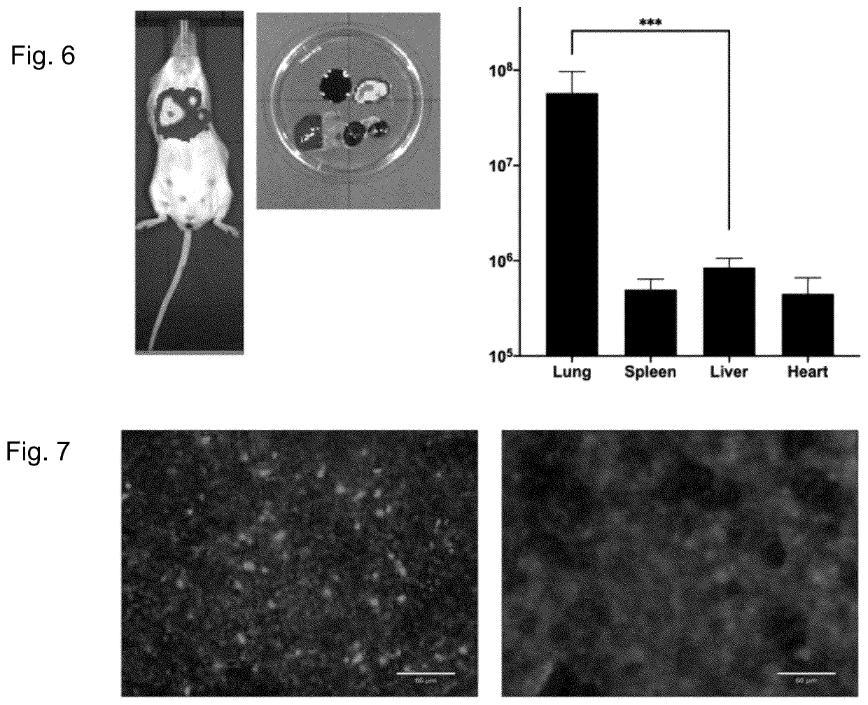

LNPs made with compound 3 deliver luciferase mRNA and CRE mRNA to the lungs with specificity. We have demonstrated that LNPs made with compound 3 can transfect lung tissue with specificity. LNPs containing various mole ratios of compound 3 were screened in mice for their ability to deliver luciferase mRNA, after an intravenous injection (10 ug mRNA per mouse). From this screen we identified an LNP formulation that contained 0.5% DMG-PEG and 40 mole % compound 3, which had exceptional selectivity for the lung. For example, LNPs containing compound 3 generated a lung signal that was 10⁸ photons/second, whereas it only generated a liver signal of approximately 10⁶ photons/second (see FIG. 6). In addition, we further validated these results via a separate CRE mRNA delivery experiment using Ai9 mice. Ai9 mice were given three consecutive injections of CRE mRNA (0.5 mg/kg per dose, 2 days apart) at 10 ug mRNA per dose, and two days after the last injection the mice were sacrificed, and the lung tissue was analyzed for fluorescence and compared against saline treated Ai9 mice (see FIG. 7). Ai9 mice treated with LNPs containing 40% of compound 3 had numerous red cells in their lung histology sections, demonstrating that LNPs made with compound 3 can transfect lung tissue. These results indicate that LNPs made with the ACE linker can deliver ASOs and CFTR mRNA to lung tissue.

1. A composition comprising azide-benzaldehyde acetal cholesterol.

2. The composition of claim 1, wherein the acetal is conjugated to PEG, wherein the azide-benzaldehyde acetal provides an acid degradable linkage between the PEG and the cholesterol.

3. The composition of claim 1, wherein the azide is reduced to an amine.

4. The composition of claim 1 of compound 1:

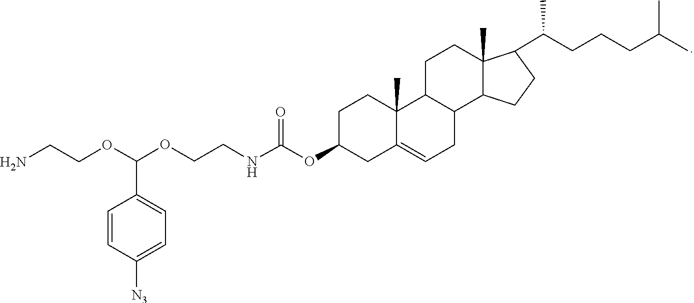 [SMILES: CC(C)CCC[C@@H](C)C1CCC2C3CC=C4C[C@@H](OC(=O)NCCOC(OCCN)c5ccc(N=[N+]=[N-])cc5)CC[C@]4(C)C3CC[C@@]21C]

5. The composition of claim 1 of compound 2:

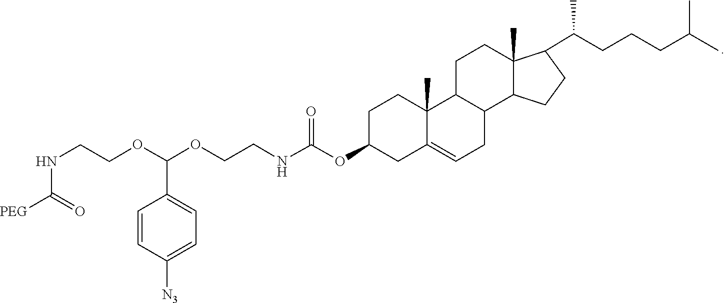

6. The composition of claim 1 of compound 3: 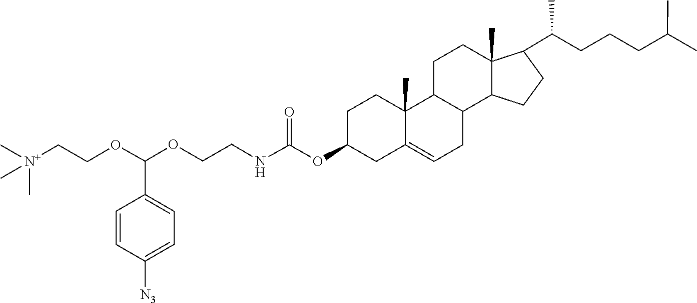 [SMILES: CC(C)CCC[C@@H](C)C1CCC2C3CC=C4C[C@@H](OC(=O)NCCOC(OCC[N+](C)(C)C)c5ccc(N=[N+]=[N-])cc5)CC[C@]4(C)C3CC[C@@]21C]
or salt thereof.

7. The composition of claim 1 formulated into solid lipid nanoparticles (SLNs).

8. The composition of claim 2 formulated into solid lipid nanoparticles (SLNs).

9. The composition of claim 3 formulated into solid lipid nanoparticles (SLNs).

10. The composition of claim 4 formulated into solid lipid nanoparticles (SLNs).

11. The composition of claim 5 formulated into solid lipid nanoparticles (SLNs).

12. The composition of claim 6 formulated into solid lipid nanoparticles (SLNs).

13. The composition of claim 1 formulated into solid lipid nanoparticles (SLNs) further comprising a nucleic acid.

14. The composition of claim 1 formulated into solid lipid nanoparticles (SLNs) further comprising a nucleic acid, that is an RNA or DNA, encoding a therapeutic protein, vaccine antigen, or gene editing enzyme.

15. The composition of claim 2 formulated into solid lipid nanoparticles (SLNs) further comprising a nucleic acid, that is an RNA or DNA, encoding a therapeutic protein, vaccine antigen, or gene editing enzyme.

16. The composition of claim 3 formulated into solid lipid nanoparticles (SLNs) further comprising a nucleic acid, that is an RNA or DNA, encoding a therapeutic protein, vaccine antigen, or gene editing enzyme.

17. The composition of claim 4 formulated into solid lipid nanoparticles (SLNs) further comprising a nucleic acid, that is an RNA or DNA, encoding a therapeutic protein, vaccine antigen, or gene editing enzyme.

18. The composition of claim 5 formulated into solid lipid nanoparticles (SLNs) further comprising a nucleic acid, that is an RNA or DNA, encoding a therapeutic protein, vaccine antigen, or gene editing enzyme.

19. The composition of claim 6 formulated into solid lipid nanoparticles (SLNs) further comprising a nucleic acid, that is an RNA or DNA, encoding a therapeutic protein, vaccine antigen, or gene editing enzyme.

20. A method of use, comprising storing and then reducing prior to biological use the composition of claim 1 formulated into solid lipid nanoparticles (SLNs), to generate an amino acetal.

21. A method of use, comprising transfecting with the composition of claim 1 a tissue or organ, such as muscle, lung, spleen, liver and blood.

22. A method of use, comprising transfecting with the composition of claim 1 a tissue or organ, such as muscle, lung, spleen, liver and blood, and detecting a resultant delivery of nucleic acid in the tissue or organ.
